SEQUENCE LISTING (4 sequences)

SEQ ID NO: 1 - DNA, 1048 bp, Bifidobacterium longum
  source 1..1048: mol_type = genomic DNA
gcttaaacatgcagtcgaacgggatccatcgggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacgggccgcaaggctaaaactcaagaaattgacgggggcccgcacaagcggcggagcatgcggattatttcgatgcaacgcgaagaaccttacctgggcttgacatgttcccgacgatcgtagagatacggcttcccttcgggcgggttcacaggtggtgcatggtcgtcgtcagctcgtgtcgtgagatgttgggttaagtcccgcaacgaggcgcaacctcg

SEQ ID NO: 2 - DNA, 960 bp, Bifidobacterium longum
  source 1..960: mol_type = genomic DNA
taccatgcagtcgaacgggatccatcgggctttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttc

SEQ ID NO: 3 - DNA, 20 bp, synthetic construct
  source 1..20: mol_type = other DNA
agagtttgatcctggctcag

SEQ ID NO: 4 - DNA, 19 bp, synthetic construct
  source 1..19: mol_type = other DNA
ggttaccttgttacgactt

(19) United States
(12) Patent Application Publication
(10) Pub. No.: US 2023/0263844 A1
(43) Pub. Date: Aug. 24, 2023

(54) Bifidobacterium longum for Preventing and/or Treating Essential Hypertension

(71) Applicant: Jiangnan University, Wuxi (CN)

(72) Inventors: Wenwei Lu, Wuxi (CN); Wei Chen, Wuxi (CN); Yusheng Wang, Wuxi (CN); Qixiao Zhai, Wuxi (CN); Jianxin Zhao, Wuxi (CH); Hao Zhang, Wuxi (CN)

(21) Appl. No.: 18/191,919

(22) Filed: Mar. 29, 2023

Related U.S. Application Data
(63) Continuation of application No. PCT/CN2021/117241, filed on Sep. 8, 2021.

(30) Foreign Application Priority Data
Sep. 30, 2020 (CN) 202011064920.1

(51) Int. Cl. A61K 35/745; A23L 33/135; A61P 9/00

(52) U.S. Cl. CPC A61K 35/745; A23L 33/135; A61P 9/00

(57) ABSTRACT

The disclosure relates to Bifidobacterium longum for preventing and/or treating essential hypertension, and belongs to the technical fields of microorganisms and medicine. The B. longum CCFM752 provided by the disclosure prevents essential hypertension, specifically including: (1) significantly decreasing the O₂⁻ level, the H₂O₂ level and the NADPH oxidase activity in A7R5 cells stimulated with Angiotensin II; (2) significantly increasing the CAT activity in A7R5 cells, aortas of SHRs and serum of SHRs; (3) significantly decreasing the blood pressure level of SHRs; (4) significantly decreasing the aortic wall ROS level, the aortic wall NADPH oxidase activity, the aortic wall collagen level and the aortic wall thickness index of SHRs; and (5) significantly increasing the aortic wall eNOS activity of SHRs. It can be seen that the B. longum CCFM752 has great application prospects in preparation of products (such as food or drugs) for preventing and/or treating hypertension.

### REFERENCE TO SEQUENCE LISTING

The instant application contains a Sequence Listing in XML format as a file named “YGHY-2022-02-seq.xml”, created on Jul. 19, 2022, of 7 kB in size, and which is hereby incorporated by reference in its entirety.

### TECHNICAL FIELD

The disclosure relates to Bifidobacterium longum for preventing and/or treating essential hypertension, and belongs to the technical fields of microorganisms and medicine.

### BACKGROUND

Hypertension is one of the most widespread chronic diseases in the population, which brings a heavy burden to the social medical system. At present, more than 30% of people in the world suffer from hypertension, and the number of people with hypertension is still increasing in recent years. More than 90% of patients with hypertension suffer from essential hypertension. Essential hypertension has a long onset period and is difficult to cure. Patients need to rely on drugs to control their blood pressure and are prone to form drug dependence. In addition, the occurrence of hypertension will greatly increase the risk of other cardiovascular diseases such as atherosclerosis, apoplexy and heart failure, and seriously threaten the life and health of patients.

The occurrence of essential hypertension mainly depends on environmental factors and living habits. Its causes are complex, and the specific mechanism is still difficult to explain. In recent years, studies have shown that reactive oxygen species (ROS) are important factors contributing to the occurrence and development of cardiovascular diseases such as hypertension. The core blood pressure regulation system of the body is the renin-angiotensin-aldosterone system (RAAS), which contains two important blood pressure-raising hormones: angiotensin II (Ang II) and aldosterone. Ang II and aldosterone can activate NADPH oxidase in the vascular wall to generate ROS such as O₂⁻ and H₂O₂ to increase the blood pressure. A dysregulation of RAAS exists in patients with essential hypertension and animal models, resulting in elevated levels of Ang II and/or aldosterone. When stimulatory factors such as Ang II and aldosterone continue to stimulate the vascular wall, excessive ROS will be generated, which will cause vasoconstriction and hypertension. The continuous production of ROS will also activate the mitogen-activated protein kinase (MAPK) pathway related to cell proliferation and the Smad pathway and TGF-β pathway related to collagen synthesis, resulting in thickening and fibrosis of the vascular wall, and causing structural lesions of the vascular wall and vascular dysfunction. Moreover, ROS in the vascular wall may also activate immune pathways such as IL-1β, IL-17 and TNF-α related to inflammation, causing vascular inflammation, and accelerating the formation of hypertension and atherosclerosis. Therefore, scavenging excessive ROS in the vascular wall will help to prevent the occurrence and development of cardiovascular diseases such as hypertension.

At present, the method of clinical intervention for hypertension is mainly drug intervention. Different types of antihypertensive drugs have toxic and side effects to different extents, such as causing electrolyte disturbances, damaging kidney function, and causing angioedema, and will damage the health of patients by long-term use. The supplementary intervention method is mainly dietary intervention. Although dietary intervention is beneficial to improve the health status of patients, the effect is slow and not significant.

The health effects of probiotics have been extensively reported in recent years, and moderate intake of probiotics has no side effects on the host. Studies have shown that some probiotics of the genus Lactobacillus have the effect of lowering blood pressure. Therefore, the use of probiotics as dietary supplements will help to improve the health status of patients and enhance the effect of non-drug interventions. However, the existing probiotics with an antihypertensive function have a single target of action, which mainly lower the blood pressure by inhibiting the angiotensin-converting enzyme (ACE) activity, and are not suitable for interfering with hypertension caused by other factors such as aldosterone. Therefore, based on the importance of ROS in the pathogenesis of hypertension, it is urgent to screen out a probiotic that can decrease the level of ROS in the vascular wall as a probiotic strain with the potential to prevent hypertension.

### SUMMARY

### Technical Problem

The disclosure is directed to the technical problem of providing a method for preventing and/or treating cardiovascular diseases.

### Technical Solution

To solve the above problem, the disclosure provides a method for preventing and/or treating cardiovascular diseases, and the method is to administer B. longum CCFM752 or a product containing B. longum CCFM752 to mammals. The B. longum CCFM752 was deposited in the Guangdong Microbial Culture Collection Center on Aug. 21, 2020, with the accession number of GDMCC No. 61157, at the 5th floor of Building 59, No. 100, Xianlie Middle Road, Guangzhou.

The B. longum CCFM752 was derived from an infant stool sample. The strain was sequenced and analyzed and its 16S rDNA sequence is shown in SEQ ID NO. 1. The sequence obtained by sequencing was compared in GeneBank for nucleic acid sequences, and the result shows that the strain belongs to B. longum and was named Bifidobacterium longum CCFM752.

The bacterial cells of the B. longum CCFM752 are in the shape of short rods, and the colonies thereof are round, raised, moist, white and glossy.

In one embodiment of the disclosure, the prevention and/or treatment of cardiovascular diseases includes the followings:
(1) significantly decreasing the O₂⁻ level and the H₂O₂ level in A7R5 cells stimulated with Angiotensin II;
(2) significantly inhibiting the NADPH oxidase activity in A7R5 cells stimulated with Angiotensin II;
(3) significantly increasing the catalase activity in A7R5 cells;
(4) significantly decreasing the blood pressure level, the aortic wall ROS level and the aortic wall NADPH oxidase activity in individuals with essential hypertension;
(5) significantly increasing the aortic wall CAT activity, the aortic wall endothelial NO synthase activity and the serum CAT activity in individuals with essential hypertension; and
(6) significantly decreasing the aortic wall collagen level and the aortic wall thickness index in individuals with essential hypertension.

In one embodiment of the disclosure, the product includes a food or a drug.

In one embodiment of the disclosure, in the product, the viable count of the B. longum CCFM752 is not less than 1×10⁶ CFU/mL or 1×10⁶ CFU/g.

In one embodiment of the disclosure, in the product, the B. longum CCFM752 is added to the food or the B. longum CCFM752 is used as a fermentation strain for food fermentation.

The disclosure further provides a drug for preventing and/or treating cardiovascular diseases, and the drug contains the B. longum CCFM752, a drug carrier and/or pharmaceutical excipients.

In one embodiment of the disclosure, the drug carrier includes microcapsules, microspheres, nanoparticles and/or liposomes.

In one embodiment of the disclosure, the pharmaceutical excipients include excipients and/or additives.

In one embodiment of the disclosure, the excipients include solvents, propellants, solubilizers, co-solvents, emulsifiers, colorants, absorbents, diluents, flocculants, deflocculants, filter aids and/or release retarders.

In one embodiment of the disclosure, the additives include microcrystalline cellulose, hydroxypropyl methylcellulose and/or refined lecithin.

In one embodiment of the disclosure, the dosage form of the drug is powder, granules, capsules, tablets, pills or oral liquid.

The disclosure further provides a food for preventing and/or treating cardiovascular diseases, and the food is a health food; or the food is a dairy product, a soy product or a fruit and vegetable product produced by using a starter containing the B. longum CCFM752; or the food is a beverage or a snack containing the B. longum CCFM752.

In one embodiment of the disclosure, the food includes solid food, liquid food and semisolid food.

In one embodiment of the disclosure, a preparation method of the starter includes: inoculating the B. longum CCFM752 into a culture medium at an inoculum of 2-4% of the total mass of the culture medium, and culturing the bacteria at 37° C. for 36 h to obtain a culture solution; centrifuging the culture solution to obtain bacterial cells; washing the bacterial cells with a phosphate buffer with a pH of 7.2-7.4 2-4 times and then resuspending the bacterial cells with a freeze-drying protective agent to obtain a resuspension; and freeze-drying the resuspension by vacuum freezing to obtain the starter.

In one embodiment of the disclosure, the mass ratio of the freeze-drying protective agent to the bacterial cells is 2:1.

In one embodiment of the disclosure, the culture medium contains 87.7% water, 10% enzymatically hydrolyzed skim milk, 0.5% glucose, 1.5% tryptone and 0.3% yeast extract of the total mass of the culture medium.

In one embodiment of the disclosure, the pH of the culture medium is 6.8.

In one example of the disclosure, the protective agent contains 100 g/L skim milk powder, 150 g/L trehalose and 10 g/L sodium L-glutamate.

Beneficial effects:

1. The disclosure provides B. longum CCFM752, and the B. longum CCFM752 can relieve hypertension, specifically by the followings:
(1) significantly decreasing the O₂⁻ level in A7R5 cells stimulated with Angiotensin II;
(2) significantly decreasing the H₂O₂ level in A7R5 cells stimulated with Angiotensin II;
(3) significantly inhibiting the NADPH oxidase activity in A7R5 cells stimulated with Angiotensin II;
(4) significantly increasing the catalase (CAT) activity in A7R5 cells;
(5) significantly decreasing the blood pressure levels in spontaneously hypertensive rats (SHRs);
(6) significantly decreasing the aortic wall ROS level of SHRs;
(7) significantly decreasing the aortic wall NADPH oxidase activity of SHRs;
(8) significantly increasing the aortic wall CAT activity of SHRs;
(9) significantly increasing the aortic wall endothelial NO synthase (eNOS) activity of SHRs;
(10) significantly increasing the serum CAT activity in SHRs;
(11) significantly decreasing the aortic wall collagen level of SHRs; and
(12) significantly decreasing the aortic wall thickness index of SHRs.

It can be seen that the B. longum CCFM752 has great application prospects in preparation of products (such as food or drugs) for preventing and/or treating hypertension.

2. B. longum is a probiotic, has been included in the “List of Bacteria that Can Be Used in Food” issued by the Ministry of Health, and has an effect of regulating intestinal health. Therefore, the B. longum CCFM752 obtained by the disclosure is relatively healthy and has no side effects to the human health.

### Biomaterial Preservation

B. longum CCFM752, named B. longum taxonomically, has been deposited in the Guangdong Microbial Culture Collection Center on Aug. 21, 2020, with the accession number of GDMCC No.61157, at the 5th floor of Building 59, No. 100, Xianlie Middle Road, Guangzhou.

### BRIEF DESCRIPTION OF FIGURES

FIG. 1 shows comparison of the O₂⁻ levels in A7R5 cells of different groups.

FIG. 2 shows comparison of the H₂O₂ levels in A7R5 cells of different groups.

FIG. 3 shows comparison of the NADPH oxidases activities in A7R5 cells of different groups.

FIG. 4 shows comparison of the CAT activities of in A7R5 cells of different groups.

FIG. 5 shows changes of systolic blood pressure of rats in different groups with age.

FIG. 6 shows changes of diastolic blood pressure of rats in different groups with age.

FIG. 7 shows comparison of ROS levels in thoracic aortic tissues of rats in different groups.

FIG. 8 shows comparison of the NADPH oxidases activities in thoracic aortic tissues of rats in different groups.

FIG. 9 shows comparison of the CAT activities in thoracic aortic tissues of rats in different groups.

FIG. 10 shows comparison of the eNOS activity in thoracic aortic tissues of rats in different groups.

FIG. 11 shows comparison of the serum CAT activities of rats in different groups.

FIG. 12 shows scanning pictures of picrosirius red stained paraffin sections of the thoracic aortas of rats in different groups.

FIG. 13 shows comparison of the collagen levels in thoracic aortic tissues of rats in different groups.

FIG. 14 shows comparison of thickness indexes of the thoracic aorta vascular walls of rats in different groups.

“##” indicates a significant difference compared with the Control group (p<0.01), “*” indicates a significant difference compared with the Model group (p<0.05), and “**” indicates a significant difference compared with the Model group (p<0.01).

### DETAILED DESCRIPTION

In the following examples, trypsin and HBSS buffers were purchased from Thermo Fisher Company; Angiotensin II was purchased from MCE Company; skim milk was purchased from Bright Dairy&Food Co., Ltd; glucose and yeast extract were purchased from Sinopharm Chemical Reagent Co., Ltd.; tryptone was purchased from OXOID Company, UK; DHE fluorescent probes, DCFH-DA fluorescent probes, DAPI staining solutions, BCA protein concentration determination kits and protease inhibitor mixtures were purchased from Beyotime Institute of Biotechnology; lucigenin was purchased from MCE Company, USA; NADPH tetrasodium and picrosirius red were purchased from Solarbio Company; and CAT assay kits and nitric oxide synthase (NOS) typing test kits were purchased from Nanjing Jiancheng Bioengineering Institute.

### The Culture Media in the Following Examples Are as Follows

An MRS liquid medium contains peptone 10 g/L, beef extract 10 g/L, glucose 20 g/L, sodium acetate 2 g/L, yeast powder 5 g/L, diammonium hydrogen citrate 2 g/L, K₂PO₄·3H₂O 2.6 g/L, MgSO₄·7H₂O 0.1 g/L, MnSO₄ 0.05 g/L, and Tween-80 1 mL/L, pH 7.0.

An MRS solid medium contains peptone 10 g/L, beef extract 10 g/L, glucose 20 g/L, sodium acetate 2 g/L, yeast powder 5 g/L, diammonium hydrogen citrate 2 g/L, K₂PO₄·3H₂O 2.6 g/L, MgSO₄·7H₂O 0.1 g/L, MnSO₄ 0.05 g/L, Tween-80 1 mL/L, agar 20 g/L, and L-cysteine hydrochloride 0.05 g/L, pH 7.0.

A DMEM medium contains glycine 30 mg/L, L-arginine hydrochloride 84 mg/L, L-cysteine hydrochloride 63 mg/L, L-glutamine 584 mg/L, L-histidine hydrochloride 42 mg/L, L-isoleucine 105 mg/L, L-leucine 105 mg/L, L-lysine hydrochloride 146 mg/L, L-methionine 30 mg/L, L-phenylalanine 66 mg/L, L-serine 42 mg/L, L-threonine 95 mg/L, L-tryptophan 16 mg/L, disodium L-tyrosine 104 mg/L, L-valine 94 mg/L, choline chloride 4 mg/L, D-calcium pantothenate 4 mg/L, folic acid 4 mg/L, nicotinamide 4 mg/L, pyridoxine hydrochloride 4 mg/L, riboflavin 0.4 mg/L, thiamine hydrochloride 4 mg/L, i-inositol 7.2 mg/L, CaCl₂ 200 mg/L, Fe(NO₃)₃·9H₂O 0.1 mg/L, MgSO₄ 97.67 mg/L, KCI 400 mg/L, NaHCO₃ 3700 mg/L, NaCl 6400 mg/L, NaH₂PO₄·H₂O 125 mg/L, D-glucose 4500 mg/L, and phenol red 15 mg/L.

### A Detection Method in the Following Examples Is as Follows

A detection method of viable count uses the national standard “GB 4789.35-2016 National Food Safety Standard, Food Microbiology Testing, Lactic Acid Bacteria Testing”.

### A Preparation Method of the Culture Supernatant and Bacterial Suspension of the B Longum in the Following Examples is as Follows

B. longum is streaked on an MRS solid medium and cultured at 37° C. for 48 h to obtain a single colony. The single colony is picked and inoculated in an MRS liquid medium, cultured at 37° C. for 18 h for activation, and activated for two consecutive generations to obtain an activated solution. The activated solution is inoculated into an MRS liquid medium at an inoculation amount of 2% (v/v) and cultured at 37° C. for 18 h to obtain a bacterial solution. The bacterial solution is centrifuged at 8000 g for 10 min to isolate the bacterial cells from the supernatant. The supernatant is adjusted to pH 7.0 with an NaOH solution of 1 mol/L, and then filtered and sterilized through a 0.22 µm filter membrane to obtain the culture supernatant of the B. longum. The bacterial cells are washed twice with a PBS buffer and then resuspended with a PBS buffer containing 30% sucrose to obtain a bacterial suspension, which is preserved in a -80° C. refrigerator for use.

### Example 1: Acquisition of B. Longum

Specific steps are as follows:

### 1. Screening

Infant feces were used as a sample, and the sample was pretreated and preserved in 20% glycerol in a -80° C. refrigerator. After the sample was taken out and thawed, the sample was mixed, and 0.5 mL of sample was pipetted and added to 4.5 mL of physiological saline. The sample was subjected to gradient dilution with 9 g/L physiological saline containing 0.05 g/L cysteine. An appropriate gradient dilution was selected and applied to an MRS solid medium and cultured at 37° C. for 48 h. A typical colony of the B. longum was picked and streaked on an MRS solid medium for purification. A single colony was picked and transferred to an MRS liquid medium (containing 0.05 g/L cysteine) for enrichment, and the bacteria were preserved in 30% glycerol to obtain strain CCFM752 and strain CCFM666, wherein the typical colonies of the B. longum are round, raised, moist, white and glossy.

### 2. Identification

The genomes of the strain CCFM752 and the strain CCFM666 were extracted, and the 16S rDNAs of the strain CCFM752 and the strain CCFM666 were amplified and sequenced (by Suzhou Jinweizhi Biotechnology Co., Ltd., wherein the determined 16S rDNA nucleotide sequences of the CCFM752 and the CCFM666 are shown in SEQ ID NO.1 and SEQ ID NO.2, the sequence of a sense primer 27F for bacterial species identification is shown in SEQ ID NO.3, and the sequence of an antisense primer 1492R is shown in SEQ ID NO.4). The sequences were compared in NCBI for nucleic acid sequences, and the results showed that both the strain CCFM752 and the strain CCFM666 were B. longum, named B. longum CCFM752 and B. longum CCFM666 respectively.

### Example 2: Effect of B. Longum on O₂⁻ level in A7R5 Cells

Specific steps are as follows:

Rat thoracic aortic smooth muscle cells A7R5 were purchased from the Cell Bank of the Committee on Type Culture Collection of Chinese Academy of Sciences, and cultured in a DMEM medium containing 10% (v/v) fetal bovine serum (FBS), 100 U/mL penicillin, and 100 mg/mL streptomycin. The cells were cultured at 37° C. in a cell incubator containing 5% (v/v) CO₂ in the gas phase, and passaged when reaching a density of 70% to 80%.

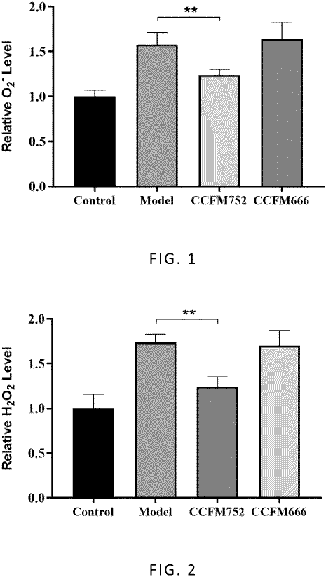

The A7R5 cells in a good growth state were selected, digested with trypsin, centrifuged, resuspended in a DMEM medium, and counted to obtain a resuspension. The resuspension was inoculated into a 24-well plate at 20,000 cells/well, 500 µL of DMEM media containing 10% (v/v) fetal bovine serum (FBS), 100 U/mL penicillin, and 100 mg/mL streptomycin was added to the 24-well plate, and the cells were cultured at 37° C. in the cell incubator containing 5% (v/v) CO₂ in the gas phase for 48 h. After 48 h of culture, the DMEM medium containing 10% (v/v) fetal bovine serum (FBS), 100 U/mL penicillin, and 100 mg/mL streptomycin in the 24-well plate was replaced with 500 µL of DMEM media containing 0.1% (v/v) fetal bovine serum (FBS), and the 24-well plate was allowed to stand at 37° C. in the cell incubator containing 5% (v/v) CO₂ in the gas phase for 24 h. After standing for 24 h, using each well of cells in the 24-well plate as the unit, the cells were divided into a blank control group (Control), a model group (Model), a B. longum CCFM752 intervention group (CCFM752) and a B. longum CCFM666 intervention group (CCFM666), with 3 wells in each group. 15 µL of MRS liquid media was added to each well of the blank control group and the model group respectively, 15 µL of B. longum CCFM752 culture supernatant was added to each well of the CCFM752 intervention group, 15 µL of B. longum CCFM666 culture supernatant was added to each well of the CCFM666 intervention group, and the 24-well plate was put at 37° C. in the cell incubator containing 5% (v/v) CO₂ in the gas phase for 12 h. After 12 h of intervention, Angiotensin II was added to each well of the model group, the CCFM752 intervention group and the CCFM666 intervention group respectively to a concentration of 1×10⁻⁷ M, the same volume of DMEM medium was added to each well of the blank control group as a control, and the 24-well plate was allowed to stand at 37° C. in the cell incubator containing 5% (v/v) CO₂ in the gas phase for 4 h. After standing for 4 h, the 24-well plate was taken out of the cell incubator, the liquid in each well was pipetted, 500 µL of DMEM media containing 10 µM DHE fluorescent probe was added to each well, and the 24-well plate was incubated at 37° C. in the cell incubator containing 5% (v/v) CO₂ in the gas phase for 30 min. After 30 min of incubation, the 24-well plate was taken out of the cell incubator, the liquid in each well was pipetted, and the cells in each well were washed 2 times with an HBSS buffer. After washing, 500 µL of the HBSS buffer was added to each well of the 24-well plate, and an inverted fluorescence microscope was used for observation, where the cells were excited to produce red fluorescence by green band excitation light, and a suitable field of view was selected for shooting. The fluorescence densities in the pictures were calculated by using Image pro plus to characterize the intracellular O₂⁻ level, and the blank control group was used as a comparison to process the data. The results are shown in FIG. 1.

From FIG. 1, after stimulation with the 1×10⁻⁷ M Angiotensin II for 4 h, the O₂⁻ level in the A7R5 cells of the model group significantly increased to 1.6 times that of the blank control group (p<0.01), and the O₂⁻ level in the A7R5 cells of the CCFM752 intervention group decreased by 27% (p<0.01) compared with the model group, while the O₂⁻ level in the A7R5 cells of the CCFM666 intervention group did not change significantly compared with the model group.

It can be seen that intervention of the B. longum CCFM752 culture supernatant can significantly inhibit the increase of the O₂⁻ level in the A7R5 cells stimulated with the Angiotensin II.

### Example 3: Effect of B. Longum on H₂O₂ level in A7R5 Cells

Specific steps are as follows:

Rat thoracic aortic smooth muscle cells A7R5 were purchased from the Cell Bank of the Committee on Type Culture Collection of Chinese Academy of Sciences, and cultured in a DMEM medium containing 10% (v/v) fetal bovine serum (FBS), 100 U/mL penicillin, and 100 mg/mL streptomycin. The cells were cultured at 37° C. in a cell incubator containing 5% (v/v) CO₂ in the gas phase, and passaged when reaching a density of 70% to 80%.

The A7R5 cells in a good growth state were selected, digested with trypsin, centrifuged, resuspended in a DMEM medium, and counted to obtain a resuspension. The resuspension was inoculated into a 24-well plate at 20,000 cells/well, 500 µL of DMEM media containing 10% (v/v) fetal bovine serum (FBS), 100 U/mL penicillin, and 100 mg/mL streptomycin was added to the 24-well plate, and the cells were cultured at 37° C. in the cell incubator containing 5% (v/v) CO₂ in the gas phase for 48 h. After 48 h of culture, the DMEM medium containing 10% (v/v) fetal bovine serum (FBS), 100 U/mL penicillin, and 100 mg/mL streptomycin in the 24-well plate was replaced with 500 µL of DMEM media containing 0.1% (v/v) fetal bovine serum (FBS), and the 24-well plate was allowed to stand at 37° C. in the cell incubator containing 5% (v/v) CO₂ in the gas phase for 24 h. After standing for 24 h, using each well of cells in the 24-well plate as the unit, the cells were divided into a blank control group (Control), a model group (Model), a B. longum CCFM752 intervention group (CCFM752) and a B. longum CCFM666 intervention group (CCFM666), with 3 wells in each group. 15 µL of MRS liquid media was added to each well of the blank control group and the model group respectively, 15 µL of B. longum CCFM752 culture supernatant was added to each well of the CCFM752 intervention group, 15 µL of B. longum CCFM666 culture supernatant was added to each well of the CCFM666 intervention group, and the 24-well plate was put at 37° C. in the cell incubator containing 5% (v/v) CO₂ in the gas phase for 12 h. After 12 h of intervention, Angiotensin II was added to each well of the model group, the CCFM752 intervention group and the CCFM666 intervention group respectively to a concentration of 1×10⁻⁷ M, the same volume of DMEM medium was added to each well of the blank control group as a control, and the 24-well plate was allowed to stand at 37° C. in the cell incubator containing 5% (v/v) CO₂ in the gas phase for 3 h. After standing for 3 h, the 24-well plate was taken out of the cell incubator, the liquid in each well was pipetted, 500 µL of DMEM media containing 8 µM DCFH-DA fluorescent probe was added to each well, and the 24-well plate was incubated at 37° C. in the cell incubator containing 5% (v/v) CO₂ in the gas phase for 20 min. After 20 min of incubation, the 24-well plate was taken out of the cell incubator, the liquid in each well was pipetted, and the cells in each well were washed 2 times with an HBSS buffer. After washing, 500 µL of the HBSS buffer was added to each well of the 24-well plate, and an inverted fluorescence microscope was used for observation, where the cells were excited to produce green fluorescence by blue band excitation light, and a suitable field of view was selected for shooting. The fluorescence densities in the pictures were calculated by using Image pro plus to characterize the intracellular H₂O₂ level, and the blank control group was used as a comparison to process the data. The results are shown in FIG. 2.

From FIG. 2, after stimulation with the 1×10⁻⁷ M Angiotensin II for 3 h, the H₂O₂ level in the A7R5 cells of the model group significantly increased to 1.8 times that of the blank control group (p<0.001), and the H₂O₂ level in the A7R5 cells of the CCFM752 intervention group decreased by 30% (p<0.01) compared with the model group, while the H₂O₂ level in the A7R5 cells of the CCFM666 intervention group did not change significantly compared with the model group.

It can be seen that intervention of the B. longum CCFM752 culture supernatant can significantly inhibit the increase of the H₂O₂ level in the A7R5 cells stimulated with the Angiotensin II.

### Example 4: Effect of B. Longum on NADPH Oxidase Activity in A7R5 Cells

Specific steps are as follows:

Rat thoracic aortic smooth muscle cells A7R5 were purchased from the Cell Bank of the Committee on Type Culture Collection of Chinese Academy of Sciences, and cultured in a DMEM medium containing 10% (v/v) fetal bovine serum (FBS), 100 U/mL penicillin, and 100 mg/mL streptomycin. The cells were cultured at 37° C. in a cell incubator containing 5% (v/v) CO₂ in the gas phase, and passaged when reaching a density of 70% to 80%.

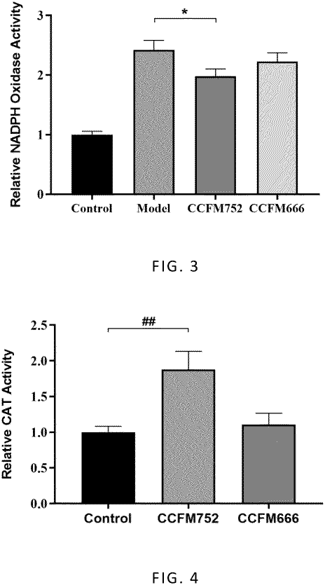

The A7R5 cells in a good growth state were selected, digested with trypsin, centrifuged, resuspended in a DMEM medium, and counted to obtain a resuspension. The resuspension was inoculated into a 6 cm cell culture dish at 2×10⁵ cells/well, 4 mL of DMEM media containing 10% (v/v) fetal bovine serum (FBS), 100 U/mL penicillin, and 100 mg/mL streptomycin was added to each cell culture dish, and the cells were cultured at 37° C. in the cell incubator containing 5% (v/v) CO₂ in the gas phase for 48 h. After 48 h of culture, the DMEM medium containing 10% (v/v) fetal bovine serum (FBS), 100 U/mL penicillin, and 100 mg/mL streptomycin in each cell culture dish was replaced with 4 mL of DMEM media containing 0.1% (v/v) fetal bovine serum (FBS), and the cell culture dishes were allowed to stand at 37° C. in the cell incubator containing 5% (v/v) CO₂ in the gas phase for 24 h. After standing for 24 h, using each dish of cells as the unit, the cells were divided into a blank control group (Control), a model group (Model), a B. longum CCFM752 intervention group (CCFM752) and a B. longum CCFM666 intervention group (CCFM666), with 3 dishes in each group. 120 µL of MRS liquid media was added to each dish of the blank control group and the model group respectively, 120 µL of B. longum CCFM752 culture supernatant was added to each dish of the CCFM752 intervention group, 120 µL of B. longum CCFM666 culture supernatant was added to each dish of the CCFM666 intervention group, and the cell culture dishes were put at 37° C. in the cell incubator containing 5% (v/v) CO₂ in the gas phase for 12 h. After 12 h of intervention, Angiotensin II was added to each dish of the model group, the CCFM752 intervention group and the CCFM666 intervention group respectively to a concentration of 1×10⁻⁷ M, the same volume of DMEM medium was added to each dish of the blank control group as a control, and the cell culture dishes were allowed to stand at 37° C. in the cell incubator containing 5% (v/v) CO₂ in the gas phase for 4 h. After standing for 4 h, the cell culture dishes were taken out of the cell incubator, and the liquid in each dish was pipetted. Afterwards the dishes were placed on ice, and washed 5 times with an ice-bath pre-cooled PBS buffer. After washing, the cells in each dish were transferred to different 15 mL centrifuge tubes, and washed 2 times with the ice-bath pre-cooled PBS buffer. After washing, PBS buffer (1 mL/tube) containing a 2.0% (v/v) protease inhibitor mixture was added to the centrifuge tubes to resuspend the cells to obtain a cell suspension. The cell suspension was transferred to a new sterile 1.5 mL centrifuge tube and placed on an ice bath for performing ultrasonic disruption (at an ultrasonic power of 300 W, for 3-5 s every 30 s, 3-5 times), to obtain a cell disruption suspension. The cell disruption solution was observed under a microscope, and was confirmed that the disruption was complete if no intact cells were observed, to complete preparation of a cell homogenate. The protein concentration of the cell homogenate was determined by a Beyotime BCA protein concentration assay kit, and the NADPH oxidase activity of the cell homogenate was determined by chemiluminescence assay: 180 µL of 50 mM phosphate buffer (pH 7.0) was added to a 96-well plate; 1 mmol/L EGTA, 150 mmol/L sucrose, 500 µmol/L lucigenin, and 100 µmol/L NADPH were added to the phosphate buffer; 5 parallel assays were set up for each cell homogenate sample; 20 µL of cell homogenate was added to each well of the 96-well plate; immediately after the cell homogenate was added, chemiluminescence assay was performed using a microplate reader; the chemiluminescence intensity was detected 30 to 120 s after the reaction started; the relative NADPH oxidase activity of the cell homogenate was characterized by the ratio of the luminescence intensity measured in each well to the protein concentration of the homogenate; and the control group was used as a comparison to process the data. The results are shown in FIG. 3.

From FIG. 3, after stimulation with the 1×10⁻⁷ M Angiotensin II for 4 h, the NADPH oxidase activity in the A7R5 cells of the model group significantly increased to 2.4 times that of the blank control group (p<0.001), and the NADPH oxidase activity in the A7R5 cells of the CCFM752 intervention group decreased by 18% (p<0.05) compared with the model group, while the NADPH oxidase activity in the A7R5 cells of the CCFM666 intervention group did not change significantly compared with the model group.

It can be seen that intervention of the B. longum CCFM752 culture supernatant can significantly inhibit the NADPH oxidase activity in the A7R5 cells stimulated with the Angiotensin II and reduce production of ROS.

### Example 5: Effect of B. Longum on CAT Activity in A7R5 Cells

Specific steps are as follows:

Rat thoracic aortic smooth muscle cells A7R5 were purchased from the Cell Bank of the Committee on Type Culture Collection of Chinese Academy of Sciences, and cultured in a DMEM medium containing 10% (v/v) fetal bovine serum (FBS), 100 U/mL penicillin, and 100 mg/mL streptomycin. The cells were cultured at 37° C. in a cell incubator containing 5% (v/v) CO₂ in the gas phase, and passaged when reaching a density of 70% to 80%.

The A7R5 cells in a good growth state were selected, digested with trypsin, centrifuged, resuspended in a DMEM medium, and counted to obtain a resuspension. The resuspension was inoculated into a 6 cm cell culture dish at 2×10⁵ cells/well, 4 mL of DMEM media containing 10% (v/v) fetal bovine serum (FBS), 100 U/mL penicillin, and 100 mg/mL streptomycin was added to each cell culture dish, and the cells were cultured at 37° C. in the cell incubator containing 5% (v/v) CO₂ in the gas phase for 48 h. After 48 h of culture, the DMEM medium containing 10% (v/v) fetal bovine serum (FBS), 100 U/mL penicillin, and 100 mg/mL streptomycin in each cell culture dish was replaced with 4 mL of DMEM media containing 0.1% (v/v) fetal bovine serum (FBS), and the cell culture dishes were allowed to stand at 37° C. in the cell incubator containing 5% (v/v) CO₂ in the gas phase for 24 h. After standing for 24 h, using each dish of cells as the unit, the cells were divided into a blank control group (Control), a model group (Model), a B. longum CCFM752 intervention group (CCFM752) and a B. longum CCFM666 intervention group (CCFM666), with 3 dishes in each group. 120 µL of MRS liquid media was added to each dish of the blank control group and the model group respectively, 120 µL of B. longum CCFM752 culture supernatant was added to each dish of the CCFM752 intervention group, 120 µL of B. longum CCFM666 culture supernatant was added to each dish of the CCFM666 intervention group, and the cell culture dishes were put at 37° C. in the cell incubator containing 5% (v/v) CO₂ in the gas phase for 12 h. After 12 h of intervention, the cell culture dishes were taken out of the cell incubator, and the liquid in each dish was pipetted, placed on ice, and washed 5 times with an ice-bath pre-cooled PBS buffer. After washing, the cells in each dish were transferred to different 15 mL centrifuge tubes, and washed 2 times with the ice-bath pre-cooled PBS buffer. After washing, PBS buffer (1 mL/tube) containing a 2.0% (v/v) protease inhibitor mixture was added to the centrifuge tubes to resuspend the cells to obtain a cell suspension. The cell suspension was transferred to a new sterile 1.5 mL centrifuge tube and placed on an ice bath for performing ultrasonic disruption (at an ultrasonic power of 300 W, for 3-5 s every 30 s, 3-5 times), to obtain a cell disruption suspension. The cell disruption suspension was observed under a microscope, and was confirmed that the disruption was complete if no intact cells were observed, to complete preparation of a cell homogenate. The protein concentration of the cell homogenate was determined by a Beyotime BCA protein concentration assay kit, the CAT activity of the cell homogenate was determined by a Nanjing Jiancheng catalase assay kit, the intracellular CAT activity was expressed by dividing the CAT activity by the protein concentration of the homogenate, and the control group was used as a comparison to process the data. The results are shown in FIG. 4.

From FIG. 4, intervention of the B. longum CCFM752 culture supernatant could increase the CAT activity in the A7R5 cells to about 1.9 times that of the blank control group (p<0.01), while intervention of the B. longum CCFM666 culture supernatant could not significantly change the CAT activity in the A7R5 cells.

It can be seen that intervention of the B. longum CCFM752 culture supernatant can help to scavenge intracellular ROS by increasing the CAT activity in the A7R5 cells.

### Example 6: Effect of B. Longum on Blood Pressure Level of SHRs

Specific steps are as follows:

4-week-old SPF grade Wistar Kyoto rats (WKYs) and spontaneously hypertensive rats (SHRs) were purchased from Beijing Vital River Laboratory Animal Co., Ltd. The laboratory animals were kept in the Laboratory Animal Center of Jiangnan University. Animal feeding and experimental conditions refer to the ethical review of the Laboratory Animal Center of Jiangnan University, approval number: JN.No20200630SW0720909[117].

SHRs were randomly divided into 3 groups: a hypertensive model group (Model), a B. longum CCFM752 intervention group (CCFM752) and a B. longum CCFM666 intervention group (CCFM666), with 7 rats in each group. All WKYs were in a normotensive control group (Control), with a total of 7 animals.

The experimental period lasted for 13 weeks in total. The first week was the adaptation period, and gavage intervention began from the second week until the end of the experiment. The CCFM752 intervention group was given a B. longum CCFM752 bacterial suspension by gavage every day at a dose of 1.0×10⁹ CFU. The CCFM666 intervention group was given a B. longum CCFM666 bacterial suspension by gavage every day at a dose of 1.0×10⁹ CFU. The normotensive control group and the hypertensive model group were not intervened with any viable bacteria or drugs, and given the same volume of PBS buffer by gavage every day as a control. All groups had free access to food and water. After the experiment, all the rats were anesthetized and sacrificed by exsanguination.

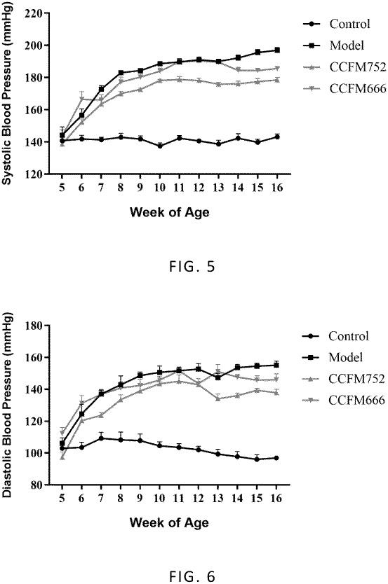

The tail artery systolic and diastolic blood pressure levels were measured once a week since the second week of the experiment (5-week-old), and the results are shown in FIGS. 5 and 6.

From FIG. 5, intervention of the viable B. longum CCFM752 could significantly decrease the systolic blood pressure of SHRs and delay the formation of hypertension, the systolic blood pressure was decreased by 9.65% compared with the hypertensive model group, while the B. longum CCFM666 could only slightly decrease the systolic blood pressure level, and did not delay the formation of essential hypertension. From FIG. 6, intervention of the viable B. longum CCFM752 could significantly decrease the diastolic blood pressure of SHRs and delay the formation of hypertension, the diastolic blood pressure was decreased by 10.97% compared with the hypertensive model group, while the B. longum CCFM666 could not significantly decrease the diastolic blood pressure, nor could it delay the formation of essential hypertension.

It can be seen that the B. longum CCFM752 can effectively prevent the occurrence and development of essential hypertension.

### Example 7: Effect of B. Longum on Aortic ROS Level of SHRs

Specific steps are as follows:

4-week-old SPF grade Wistar Kyoto rats (WKYs) and spontaneously hypertensive rats (SHRs) were purchased from Beijing Vital River Laboratory Animal Co., Ltd. The laboratory animals were kept in the Laboratory Animal Center of Jiangnan University. Animal feeding and experimental conditions refer to the ethical review of the Laboratory Animal Center of Jiangnan University, approval number: JN.No20200630SW0720909[117].

SHRs were randomly divided into 3 groups: a hypertensive model group (Model), a B. longum CCFM752 intervention group (CCFM752) and a B. longum CCFM666 intervention group (CCFM666), with 7 rats in each group. All WKYs were in a normotensive control group (Control), with a total of 7 animals.

The experimental period lasted for 13 weeks in total. The first week was the adaptation period, and gavage intervention began from the second week until the end of the experiment. The CCFM752 intervention group was given a B. longum CCFM752 bacterial suspension by gavage every day at a dose of 1.0×10⁹ CFU. The CCFM666 intervention group was given a B. longum CCFM666 bacterial suspension by gavage every day at a dose of 1.0×10⁹ CFU. The normotensive control group and the hypertensive model group were not intervened with any viable bacteria or drugs, and given the same volume of PBS buffer by gavage every day as a control. All groups had free access to food and water. After the experiment, all the rats were anesthetized and sacrificed by exsanguination, and the thoracic aortas were collected.

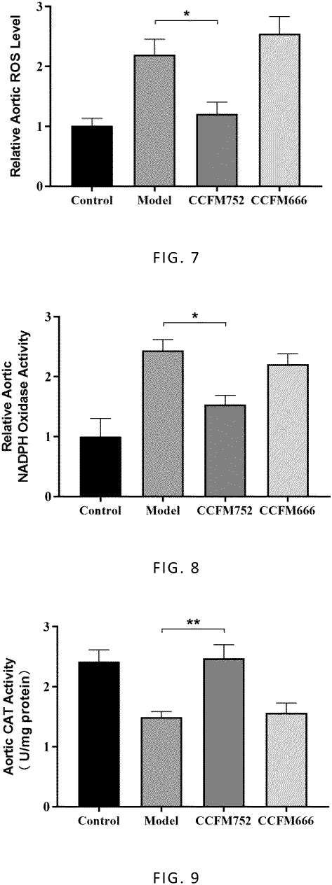

The collected aortic tissue was dehydrated in PBS (pH 7.4) containing 30% (w/w) sucrose for 1-2 h, then embedded using OCT embedding agent, and then frozen in a -80° C. refrigerator. The frozen tissue was taken out, and cut into 10 µm thick sections in a cryostat. The sections were stained in a physiological saline solution containing 5 µM DHE for 30 min, and then counterstained in a physiological saline solution containing 300 µM DAPI for 30 min. The stained sections were taken out, washed and mounted. The sections were photographed within 24 h after preparation by an inverted fluorescence microscope. The aortic ROS level was expressed as the ratio of ethidium fluorescence intensity/DAPI fluorescence intensity, and the normotensive control group was used as a comparison to process the data. The results are shown in FIG. 7.

From FIG. 7, intervention of the viable B. longum CCFM752 could significantly decrease the aortic ROS level of SHRs, and the ROS level was decreased by 55.45% compared with the hypertensive model group, while the B. longum CCFM666 could not significantly decrease the aortic ROS level.

It can be seen that the B. longum CCFM752 has an antioxidative effect on SHRs and helps to reduce the occurrence of hypertension.

### Example 8: Effect of B. Longum on Aortic NADPH Oxidase Activity of SHRs

Specific steps are as follows:

4-week-old SPF grade Wistar Kyoto rats (WKYs) and spontaneously hypertensive rats (SHRs) were purchased from Beijing Vital River Laboratory Animal Co., Ltd. The laboratory animals were kept in the Laboratory Animal Center of Jiangnan University. Animal feeding and experimental conditions refer to the ethical review of the Laboratory Animal Center of Jiangnan University, approval number: JN.No20200630SW0720909[117].

SHRs were randomly divided into 3 groups: a hypertensive model group (Model), a B. longum CCFM752 intervention group (CCFM752) and a B. longum CCFM666 intervention group (CCFM666), with 7 rats in each group. All WKYs were in a normotensive control group (Control), with a total of 7 animals.

The experimental period lasted for 13 weeks in total. The first week was the adaptation period, and gavage intervention began from the second week until the end of the experiment. The CCFM752 intervention group was given a B. longum CCFM752 bacterial suspension by gavage every day at a dose of 1.0×10⁹ CFU. The CCFM666 intervention group was given a B. longum CCFM666 bacterial suspension by gavage every day at a dose of 1.0×10⁹ CFU. The normotensive control group and the hypertensive model group were not intervened with any viable bacteria or drugs, and given the same volume of PBS buffer by gavage every day as a control. All groups had free access to food and water. After the experiment, all the rats were anesthetized and sacrificed by exsanguination, and the thoracic aortic tissue was collected.

The collected aortic tissue was cut into rings with a thickness of 3-4 mm, placed in a physiological saline solution, and temporarily preserved in an ice bath. Before the experiment, the aortic tissue rings were placed in a constant temperature incubator at 37° C. for 30 min. The aortic tissue rings were placed in a 96-well plate, 200 µL of physiological saline solutions containing 100 µM NADPH and 5 µM lucigenin was added, and then the aortic tissue rings were immediately placed in a microplate reader for determination. The chemiluminescence intensity was read 30-200 s after the reaction started. After the determination, the aortic tissue rings were taken out, dried, and determined the dry weight of the tissue, and the relative NADPH oxidase activity of the tissue was expressed by reading the total chemiluminescence intensity/tissue dry weight. The normotensive control group was used as a comparison to process the data, and the results are shown in FIG. 8.

From FIG. 8, intervention of the viable B. longum CCFM752 could decrease the aortic NADPH oxidase activity of SHRs, and the NADPH oxidase activity was decreased by 53.51% compared with the hypertensive model group, while the B. longum CCFM666 could not decrease the NADPH oxidase activity.

It can be seen that the B. longum CCFM752 can inhibit the production of ROS in the aortic tissue of SHRs and effectively reduce the risk of hypertension and vascular remodeling.

### Example 9: Effect of B. Longum on Aortic CAT Activity of SHRs

Specific steps are as follows:

4-week-old SPF grade Wistar Kyoto rats (WKYs) and spontaneously hypertensive rats (SHRs) were purchased from Beijing Vital River Laboratory Animal Co., Ltd. The laboratory animals were kept in the Laboratory Animal Center of Jiangnan University. Animal feeding and experimental conditions refer to the ethical review of the Laboratory Animal Center of Jiangnan University, approval number: JN.No20200630SW0720909[117].

SHRs were randomly divided into 3 groups: a hypertensive model group (Model), a B. longum CCFM752 intervention group (CCFM752) and a B. longum CCFM666 intervention group (CCFM666), with 7 rats in each group. All WKYs were in a normotensive control group (Control), with a total of 7 animals.

The experimental period lasted for 13 weeks in total. The first week was the adaptation period, and gavage intervention began from the second week until the end of the experiment. The CCFM752 intervention group was given a B. longum CCFM752 bacterial suspension by gavage every day at a dose of 1.0×10⁹ CFU. The CCFM666 intervention group was given a B. longum CCFM666 bacterial suspension by gavage every day at a dose of 1.0×10⁹ CFU. The normotensive control group and the hypertensive model group were not intervened with any viable bacteria or drugs, and given the same volume of PBS buffer by gavage every day as a control. All groups had free access to food and water. After the experiment, all the rats were anesthetized and sacrificed by exsanguination, and the thoracic aortic tissue was collected.

The aortic tissue was weighed, and PBS (pH 7.4, containing 2% protease inhibitor and phosphatase inhibitor) was added at a mass ratio of 1:9. The aortic tissue was minced and disrupted in a high-throughput tissue grinder after grinding beads were added (disruption conditions: the oscillation frequency was 65 Hz, the oscillation lasted for 15 s per time, and continuous disruption was performed 8-10 times.). The disrupted tissue was centrifuged at 3000×g at 4° C. for 15 min, and the supernatant was separately preserved and frozen in a -80° C. refrigerator for use. The protein concentration of the aortic tissue homogenate was determined by a BCA protein concentration assay kit, the CAT activity was determined by a Nanjing Jiancheng catalase assay kit, and the CAT activity of the aortic tissue was expressed by dividing the CAT activity of the homogenate by the protein concentration of the homogenate. The results are shown in FIG. 9.

From FIG. 9, intervention of the viable B. longum CCFM752 could improve the aortic CAT activity of SHRs, and the CAT activity was improved by 65.80% compared with the hypertensive model group, while the B. longum CCFM666 could not improve the CAT activity.

It can be seen that the B. longum CCFM752 can improve the aortic CAT activity of SHRs, improve the antioxidative capacity, and help to scavenge ROS from the vessel wall.

### Example 10: Effect of B. Longum on Aortic eNOS Activity of SHRs

Specific steps are as follows:

4-week-old SPF grade Wistar Kyoto rats (WKYs) and spontaneously hypertensive rats (SHRs) were purchased from Beijing Vital River Laboratory Animal Co., Ltd. The laboratory animals were kept in the Laboratory Animal Center of Jiangnan University. Animal feeding and experimental conditions refer to the ethical review of the Laboratory Animal Center of Jiangnan University, approval number: JN.No20200630SW0720909[117].

SHRs were randomly divided into 3 groups: a hypertensive model group (Model), a B. longum CCFM752 intervention group (CCFM752) and a B. longum CCFM666 intervention group (CCFM666), with 7 rats in each group. All WKYs were in a normotensive control group (Control), with a total of 7 animals.

The experimental period lasted for 13 weeks in total. The first week was the adaptation period, and gavage intervention began from the second week until the end of the experiment. The CCFM752 intervention group was given a B. longum CCFM752 bacterial suspension by gavage every day at a dose of 1.0×10⁹ CFU. The CCFM666 intervention group was given a B. longum CCFM666 bacterial suspension by gavage every day at a dose of 1.0×10⁹ CFU. The normotensive control group and the hypertensive model group were not intervened with any viable bacteria or drugs, and given the same volume of PBS buffer by gavage every day as a control. All groups had free access to food and water. After the experiment, all the rats were anesthetized and sacrificed by exsanguination, and the thoracic aortic tissue was collected.

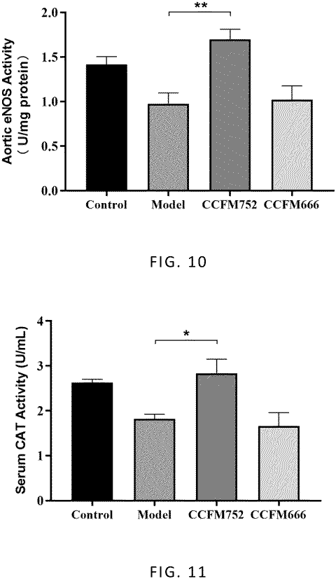

The aortic tissue was weighed, and PBS (pH 7.4, containing 2% protease inhibitor and phosphatase inhibitor) was added at a mass ratio of 1:9. The aortic tissue was minced and disrupted in a high-throughput tissue grinder after grinding beads were added. Disruption conditions were: a module was pre-cooled at -20° C., the oscillation frequency was 65 Hz, the oscillation lasted for 15 s per time, and continuous disruption was performed 8-10 times. The disrupted tissue was centrifuged at 3000×g at 4° C. for 15 min, and the supernatant was separately preserved and frozen in a -80° C. refrigerator for use. The protein concentration of the aortic tissue homogenate was determined by a BCA protein concentration assay kit, and the eNOS activity was determined by a Nanjing Jiancheng nitric oxide synthase typing kit. The aortic eNOS activity was expressed by dividing the eNOS activity of the homogenate by the protein concentration of the homogenate, and the results are shown in FIG. 10.

From FIG. 10, intervention of the viable B. longum CCFM752 could significantly improve the aortic eNOS activity of SHRs, and the eNOS activity was improved by 75.25% compared with the hypertensive model group, while the B. longum CCFM666 could not improve the eNOS activity.

It can be seen that the B. longum CCFM752 can increase the aortic endothelial NO synthesis level of SHRs, effectively promote vasodilation and improve vascular function.

### Example 11: Effect of B. Longum on Serum CAT Activity of SHRs

Specific steps are as follows:

4-week-old SPF grade Wistar Kyoto rats (WKYs) and spontaneously hypertensive rats (SHRs) were purchased from Beijing Vital River Laboratory Animal Co., Ltd. The laboratory animals were kept in the Laboratory Animal Center of Jiangnan University. Animal feeding and experimental conditions refer to the ethical review of the Laboratory Animal Center of Jiangnan University, approval number: JN.No20200630SW0720909[117].

SHRs were randomly divided into 3 groups: a hypertensive model group (Model), a B. longum CCFM752 intervention group (CCFM752) and a B. longum CCFM666 intervention group (CCFM666), with 7 rats in each group. All WKYs were in a normotensive control group (Control), with a total of 7 animals.

The experimental period lasted for 13 weeks in total. The first week was the adaptation period, and gavage intervention began from the second week until the end of the experiment. The CCFM752 intervention group was given a B. longum CCFM752 bacterial suspension by gavage every day at a dose of 1.0×10⁹ CFU. The CCFM666 intervention group was given a B. longum CCFM666 bacterial suspension by gavage every day at a dose of 1.0×10⁹ CFU. The normotensive control group and the hypertensive model group were not intervened with any viable bacteria or drugs, and given the same volume of PBS buffer by gavage every day as a control. All groups had free access to food and water. After the experiment, all the rats were anesthetized and sacrificed by exsanguination and blood was collected. The blood was centrifuged at 3000×g at 4° C. for 15 min, and then the serum was separated and preserved in a -80° C. refrigerator for use.

The serum CAT activity was measured using a Nanjing Jiancheng catalase assay kit, and the results are shown in FIG. 11.

From FIG. 11, intervention of the viable B. longum CCFM752 could significantly improve the serum CAT activity of SHRs, and the CAT activity was improved by 56.90% compared with the hypertensive model group, while the B. longum CCFM666 could not improve the serum CAT activity.

It can be seen that the B. longum CCFM752 can improve the systemic antioxidative capacity of SHRs and effectively prevent oxidative stress and oxidative damage.

### Example 12: Effect of B. Longum on Aortic Collagen Level of SHRs

Specific steps are as follows:

4-week-old SPF grade Wistar Kyoto rats (WKYs) and spontaneously hypertensive rats (SHRs) were purchased from Beijing Vital River Laboratory Animal Co., Ltd. The laboratory animals were kept in the Laboratory Animal Center of Jiangnan University. Animal feeding and experimental conditions refer to the ethical review of the Laboratory Animal Center of Jiangnan University, approval number: JN.No20200630SW0720909[117].

SHRs were randomly divided into 3 groups: a hypertensive model group (Model), a B. longum CCFM752 intervention group (CCFM752) and a B. longum CCFM666 intervention group (CCFM666), with 7 rats in each group. All WKYs were in a normotensive control group (Control), with a total of 7 animals.

The experimental period lasted for 13 weeks in total. The first week was the adaptation period, and gavage intervention began from the second week until the end of the experiment. The CCFM752 intervention group was given a B. longum CCFM752 bacterial suspension by gavage every day at a dose of 1.0×10⁹ CFU. The CCFM666 intervention group was given a B. longum CCFM666 bacterial suspension by gavage every day at a dose of 1.0×10⁹ CFU. The normotensive control group and the hypertensive model group were not intervened with any viable bacteria or drugs, and given the same volume of PBS buffer by gavage every day as a control. All groups had free access to food and water. After the experiment, all the rats were anesthetized and sacrificed by exsanguination, and the thoracic aortas were collected.

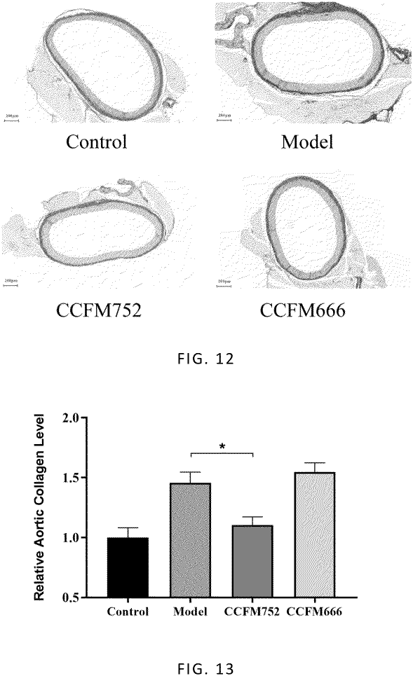

The aortas about 0.5 cm long were cut, immediately fixed in a PBS solution containing 4% paraformaldehyde, dehydrated, embedded in paraffin, and cut into 10 µm-thick sections. The tissue sections were stained in a picrosirius red staining solution (the concentration of picrosirius red was 0.1% (w/v), dissolved in a saturated picric acid aqueous solution). The picrosirius red stained sections were scanned with a digital slide scanner (shown in FIG. 12). The relative aortic collagen level was calculated using Image-Pro Plus software, and the normotensive control group was used as a comparison to process the data. The results are shown in FIG. 13.

From FIG. 13, intervention of the viable B. longum CCFM752 could significantly decrease the aortic collagen level of SHRs, and the aortic collagen level was decreased by 24.66% compared with the hypertensive model group, while the B. longum CCFM666 could not decrease the aortic collagen level.

It can be seen that the B. longum CCFM752 can effectively prevent hypertension-related vascular fibrosis and help maintain vascular elasticity.

### Example 13: Effect of B. Longum on Aortic Thickness Index of SHRs

Specific steps are as follows:

4-week-old SPF grade Wistar Kyoto rats (WKYs) and spontaneously hypertensive rats (SHRs) were purchased from Beijing Vital River Laboratory Animal Co., Ltd. The laboratory animals were kept in the Laboratory Animal Center of Jiangnan University. Animal feeding and experimental conditions refer to the ethical review of the Laboratory Animal Center of Jiangnan University, approval number: JN.No20200630SW0720909[117].

SHRs were randomly divided into 3 groups: a hypertensive model group (Model), a B. longum CCFM752 intervention group (CCFM752) and a B. longum CCFM666 intervention group (CCFM666), with 7 rats in each group. All WKYs were in a normotensive control group (Control), with a total of 7 animals.

The experimental period lasted for 13 weeks in total. The first week was the adaptation period, and gavage intervention began from the second week until the end of the experiment. The CCFM752 intervention group was given a B. longum CCFM752 bacterial suspension by gavage every day at a dose of 1.0×10⁹ CFU. The CCFM666 intervention group was given a B. longum CCFM666 bacterial suspension by gavage every day at a dose of 1.0×10⁹ CFU. The normotensive control group and the hypertensive model group were not intervened with any viable bacteria or drugs, and given the same volume of PBS buffer by gavage every day as a control. All groups had free access to food and water. After the experiment, all the rats were anesthetized and sacrificed by exsanguination, and the thoracic aortas were collected.

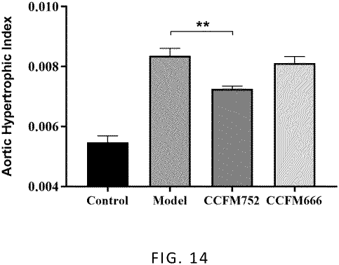

The aortas about 0.5 cm long were cut, immediately fixed in a PBS solution containing 4% paraformaldehyde, dehydrated, embedded in paraffin, and cut into 10 µm-thick sections. The tissue sections were stained in a picrosirius red staining solution (the concentration of picrosirius red was 0.1% (w/v), dissolved in a saturated picric acid aqueous solution). The picrosirius red stained sections were scanned with a digital slide scanner (shown in FIG. 12). The aortic hypertrophy and luminal circumference were measured using Image-Pro Plus software, and the aortic thickness index was expressed as the ratio of the aortic hypertrophy to the luminal circumference. The results are shown in FIG. 14.

From FIG. 14, intervention of the viable B. longum CCFM752 could significantly decrease the aortic thickness index of SHRs, and the aortic thickness index was decreased by 13.15% compared with the hypertensive model group, while the B. longum CCFM666 could not decrease the aortic thickness index.

It can be seen that the B. longum CCFM752 can effectively prevent hypertension-related aortic hypertrophy and reduce the risk of vascular stenosis.

### Example 14: Application of B. Longum

B. longum CCFM752 can be used for preparing a solid beverage by the following specific preparation process:

The B. longum CCFM752 was inoculated into a culture medium at an inoculum of 2-4% of the total mass of the culture medium and cultured at 37° C. for 36 h to obtain a bacterial suspension. The suspension was centrifuged to obtain bacterial cells. The bacterial cells were washed 2-4 times with a phosphate buffer with a pH of 7.2-7.4 and then resuspended with a freeze-drying protective agent to obtain a resuspension. The resuspension was freeze-dried by vacuum freezing to obtain bacterial powder. The mass ratio of the freeze-drying protective agent to the bacterial cells was 2:1. The culture medium contained 87.7% water, 10% enzymatically hydrolyzed skim milk, 0.5% glucose, 1.5% tryptone and 0.3% yeast extract, accounting for the total mass of the culture medium with pH 6.8. The protective agent contained 100 g/L skimmed milk powder, 150 g/L trehalose and 10 g/L sodium L-glutamate.

The solid beverage containing B. longum CCFM752 was prepared by mixing the bacterial powder containing 10¹⁰ CFU B. longum CCFM752 with 1 g of maltodextrin.

Although the disclosure has been disclosed as above in the preferred examples, it is not intended to limit the disclosure. Any person skilled in the art can make various changes and modifications without departing from the spirit and scope of the disclosure. Therefore, the protection scope of the disclosure should be as defined in the claims.

What is claimed is:

1. A method for preventing and/or treating cardiovascular diseases, comprising administering Bifidobacterium longum (B. longum) CCFM752 or a product containing B. longum CCFM752 to mammals; and the B. longum was deposited in the Guangdong Microbial Culture Collection Center (GDMCC) on August 21, 2020, with the accession number of GDMCC No. 61157.

2. The method of claim 1, wherein the prevention and/or treatment of cardiovascular diseases comprises:
(1) significantly decreasing the O₂⁻ level and the H₂O₂ level in A7R5 cells stimulated with Angiotensin II;
(2) significantly inhibiting the NADPH oxidase activity in A7R5 cells stimulated with Angiotensin II;
(3) significantly increasing the catalase activity in A7R5 cells;
(4) significantly decreasing the blood pressure level, the aortic wall ROS level and the aortic wall NADPH oxidase activity in individuals with essential hypertension;
(5) significantly increasing the aortic wall CAT activity, the aortic wall endothelial NO synthase activity and the serum CAT activity in individuals with essential hypertension; and
(6) significantly decreasing the aortic wall collagen level and the aortic wall thickness index in individuals with essential hypertension.

3. The method of claim 1, wherein the product comprises a food or a drug.

4. The method of claim 1, wherein in the product, the viable count of the B. longum CCFM752 is not less than 1×10⁶ CFU/mL or 1×10⁶ CFU/g.

5. The method of claim 3, wherein in the product, the B. longum CCFM752 is added to the food or the B. longum CCFM752 is used as a fermentation strain for food fermentation.

6. A drug for preventing and/or treating cardiovascular diseases, containing the Bifidobacterium longum (B. longum) CCFM752, a drug carrier and/or pharmaceutical excipients.

7. The drug of claim 6, wherein the drug carrier comprises microcapsules, microspheres, nanoparticles and/or liposomes.

8. The drug of claim 6, wherein the pharmaceutical excipients comprise excipients and/or additives.

9. The drug of claim 8, wherein the excipients comprise solvents, propellants, solubilizers, co-solvents, emulsifiers, colorants, absorbents, diluents, flocculants, deflocculants, filter aids and/or release retarders; and the additives comprise microcrystalline cellulose, hydroxypropyl methylcellulose and/or refined lecithin.

10. The drug of claim 6, wherein the dosage form of the drug is powder, granules, capsules, tablets, pills or oral liquid.

11. A food for preventing and/or treating cardiovascular diseases, wherein the food comprises health food; or the food is a dairy product, a soy product or a fruit and vegetable product produced by using a starter containing the Bifidobacterium longum (B. longum) CCFM752; or the food is a beverage or a snack containing the B. longum CCFM752.

12. The food of claim 11, wherein the food comprises solid food, liquid food and semi-solid food.
